(19) United States
(12) Patent Application Publication
(10) Pub. No.: US 2016/0089401 A1
(43) Pub. Date: Mar. 31, 2016

(54) USES OF TREHALOSE IN CELL SUSPENSIONS

(71) Applicant: Cook General Biotechnology LLC, Indianapolis, IN (US)

(72) Inventors: Erik John Woods, Indianapolis, IN (US); Sreedhar Thirumala, Zionsville, IN (US); Shelly J. Zacharias, Indianapolis, IN (US); Matthew B. Welty, Indianapolis, IN (US)

(21) Appl. No.: 14/869,006

(22) Filed: Sep. 29, 2015

Related U.S. Application Data
(60) Provisional application No. 62/056,842, filed on Sep. 29, 2014.

(51) Int. Cl. A61K 35/28; A61K 47/26

(52) U.S. Cl. CPC A61K 35/28; A61K 47/26

(57) ABSTRACT

Disclosed are cellular compositions and methods relating to the use of aqueous trehalose media to suspend cells. A trehalose-containing medium can be used to inhibit cellular clumping, for example upon dilution of more concentrated cellular preparations into the trehalose-containing medium. In certain embodiments cells, after cryopreservation and thawing, are combined with a trehalose-containing medium to prepare a clumping-inhibited cell suspension.

### REFERENCE TO RELATED APPLICATION

This application claims the benefit of U.S. Provisional Patent Application No. 62/056,842, filed Sep. 29, 2014 which is hereby incorporated by reference herein in its entirety.

### BACKGROUND

Aspects of the present disclosure relate generally to cellular compositions, and in particular aspects to liquid compositions useful for diluting or suspending cells previously subjected to cryopreservation.

Administration of cellular compositions to humans and animals in the treatment of various pathologies or disorders has become increasingly prevalent and bears hope to improve a multitude of therapies. The nature of the cellular composition as it is administered to the patient is important. Additives to the compositions must be biologically acceptable to the patient. As well, the state of the cells in the composition, as well as their viability before, during and after the administration protocol, are of high importance.

In one area of concern, cells suspended in liquid media often tend to aggregate or clump. Clumping has been observed as a particular problem in some cases after cells have been cryopreserved and then thawed. This can frustrate attempts to recover, retain and deliver high therapeutic doses of cells. As well, administration of highly clumped cellular preparations may pose risks to the patient that could be ameliorated with effective ways to minimize or reduce cell clumping.

In view of the background in the area, there remain needs for improved and/or alternative methods and compositions for preparing cell suspension compositions, for example that are suitable for delivery to a human or animal patient. Aspects of the present disclosure are addressed to these needs.

### SUMMARY

In certain aspects disclosed herein, it has been discovered that aqueous media containing trehalose find advantageous use in conjunction with the preparation of cell suspensions, preferably mammalian cell suspensions. Trehalose has been discovered to beneficially inhibit cell clumping, allowing for the preparation of improved cell suspensions for therapeutic or other uses. The cells can be cells previously subjected to cryopreservation and thawing. The thawed cells can be combined with an aqueous medium containing trehalose to prepare a cell suspension.

In accordance with one embodiment disclosed herein, a method for preparing a cell suspension includes the steps of (i) thawing cryopreserved cells to provide thawed cells; and (ii) combining the thawed cells with an aqueous medium containing trehalose. In preferred embodiments the combining step includes affecting a dilution of the thawed cells from a first, higher density of cells per milliliter (cells/mL) to a second, lower density of cells/mL. For example, the dilution can be cause at least a 30% reduction in the density in cells/mL, or at least a 50% reduction in the density in cells/mL. In addition or alternatively, the dilution can be from first density of greater than 3 million cells/mL to a second density of less than 2.5 million cells/mL, for example in the range of about 250,000 to about 2.5 million cells/mL.

In accordance with other embodiments disclosed herein, provided are liquid compositions for preparing a trehalose-containing cell suspension. The liquid compositions include a sterile aqueous medium containing trehalose at a concentration in the range of about 0.5% to about 20% by weight, more preferably about 0.5% to about 10% by weight, and/or having an osmolarity in the range of about 200 to about 600. The compositions can include about 0.9% sodium chloride and are desirably also buffered, for example with phosphate buffer, and have a pH of about 6 to about 8.

In accordance with still further embodiments disclosed herein, provided are kits for preparing a cell suspension that include liquid trehalose compositions as described above and elsewhere herein, in combination with a container, for example a bag, for combining the liquid trehalose composition with cells. The kits can also include a container (e.g. a vial or bag) containing the cells to be combined with the liquid trehalose composition and/or a filter through which the prepared cell suspension can be passed prior to administration of the cell suspension into the patient.

Additional embodiments, as well as features and advantages thereof, will be apparent from the descriptions herein.

### DETAILED DESCRIPTION

Reference to certain embodiments will be made in this detailed description and specific language will be used to describe the embodiments. It will be understood that this description is intended to be illustrative. Any alterations and further modifications in the described embodiments, and any further applications of the principles thereof, are contemplated as would normally occur to one skilled in the art to which this disclosure pertains.

As disclosed above, in certain aspects the present disclosure relates to methods for preparing cell suspensions that contain trehalose, and compositions and kits useful for the same.

Trehalose, also known as mycose or tremalose, is an alpha-linked disaccharide formed by an α,α-1,1-glucoside bond between two α-glucose units. It has a chemical name of (2R,3S,4S,5R,6R)-2-(hydroxymethyl)-6-[(2R,3R,4S,5S,6R)-3,4,5-trihydroxy-6-(hydroxymethyl)oxan-2-yl]oxyoxane-3,4,5-triol (IUPAC naming convention).

In certain embodiments herein, an aqueous medium (e.g. solution) containing trehalose is combined with cells to prepare a cell suspension. The aqueous medium can contain any suitable concentration of trehalose for these purposes. In certain aspects, the aqueous medium contains trehalose at a concentration of about 1% to about 20% by weight, or about 1% to about 10% by weight, or about 2% to about 7% by weight, or about 2.5% to about 5% by weight, or about 3% to about 4% by weight. The aqueous medium can also include other components. For example, it can include sodium chloride at a physiologically acceptable level, for example at a level in the range of about 0.5% to about 1.5%, e.g. about 0.9% (isotonic). The aqueous medium can also include a buffer, for example phosphate buffer, and can have a pH in the range of about 6 to 8, or about 6.8 to 7.8, or about 7 to 7.5. The aqueous medium can also have an osmolarity in the range of 200 to 600 milliosmols per kilogram (mosm/kg), or 250 to 500 mosm/kg, or 250 to 400 mosm/kg.

The aqueous medium can be combined with the cells to prepare a trehalose-containing cell suspension having a suitable concentration of trehalose. This concentration of trehalose, in certain embodiments, is in the range of about 1% to about 10% by weight trehalose or about 2% to about 7% by weight, or about 2.5% to about 5% by weight, or about 3% to about 4% by weight. Additionally or alternatively, the concentration of trehalose can be effective to inhibit clumping of the cells as compared to a corresponding cell suspension without the trehalose. Inhibition of clumping can be observed by the formation of fewer and/or smaller clumps of cells in the prepared cell suspension, for example at a time point at least ten minutes after preparation of the cell suspension, at least twenty minutes after preparation of the cell suspension, or at least after 60 minutes after preparation of the cell suspension. The capacity of the trehalose to inhibit clumping for significant periods of time following preparation of the cell suspension can, for example, provide sufficient time to administer the prepared cell suspension to a patient, for example by injecting or infusing the cell suspension into the bloodstream of a patient by venous or arterial access and/or by local implantation of the cell suspension. In therapeutic applications of cell suspensions, the suspension can be administered to the patient over a relatively prolonged period of time, for example at least 10 minutes, at least 20 minutes, or at least 60 minutes.

The cell composition that is used in the preparation of the trehalose-containing cell suspensions disclosed in this document can be any suitable cell composition. In certain embodiments, the cell composition will be one that has been cryopreserved. Such cryopreservation can in certain aspects involved freezing the cell composition at a temperature of −60° C. or lower, of −80° C. or lower, and in certain aspects at a temperature of about −196° C. (e.g. in liquid nitrogen). Cryopreserved cell compositions will typically include a cryoprotectant agent, for example dimethyl sulfoxide (DMSO), glycerol, lactose-egg yolk extender, trehalose and/or other such agents. In certain embodiments the cryopreserved cell compositions will be trehalo se-free or essentially trehalose-free (i.e. containing less than about 0.1% trehalose). The cell density of the cryopreserved or other cell composition to be used can vary. In some forms, the cell density will be at least about 1 million cells/mL, at least 2 million cells/mL, or at least 5 million cells/mL, and typically in the range of 1 million cells/mL to 100 million cells/mL, more typically in the range of 1 million cells/mL to 20 million cells/mL. When cryopreserved, the cells can be sealed within a suitable container such as a cryobag or cryovial, constructed to withstand the conditions of the cryopreservation while maintaining the integrity of the container seal.

A wide variety of cell types may be used in embodiments of the present disclosure. For example, the cells can be skin cells, skeletal muscle cells, cardiac muscle cells, lung cells, mesentery cells, adipose cells, or stem cells such as mesenchymal stem cells. Adipose cells may be from omental fat, properitoneal fat, perirenal fat, pericardial fat, subcutaneous fat, breast fat, or epididymal fat. In certain embodiments, the cells comprise stromal cells, stem cells, or combinations thereof. As used herein, the term “stem cells” is used in a broad sense and includes traditional stem cells, adipose derived stem cells, progenitor cells, preprogenitor cells, reserve cells, and the like. Exemplary stem cells include embryonic stem cells, adult stem cells, pluripotent stem cells, neural stem cells, liver stem cells, muscle stem cells, muscle precursor stem cells, endothelial progenitor cells, bone marrow stem cells, chondrogenic stem cells, lymphoid stem cells, mesenchymal stem cells, hematopoietic stem cells, central nervous system stem cells, peripheral nervous system stem cells, and the like. Additional illustrative cells which can be used include hepatocytes, epithelial cells, Kupffer cells, fibroblasts, neurons, cardiomyocytes, myocytes, chondrocytes, pancreatic acinar cells, islets of Langerhans, osteocytes, myoblasts, satellite cells, endothelial cells, adipocytes, preadipocytes, biliary epithelial cells, and progentior cells of any of these cell types.

When used, mesenchymal stem cells (MSC) can be obtained from any suitable tissue. These include as examples MSCs derived from dental tissue (such as those harvested from dental pulp, periodontal ligaments, or other dental tissues), testicle tissue, bone marrow; peripheral blood, placental tissue, uterine tissue (including endometrial regenerative cells), umbilical cord blood, umbilical cord tissue, or skin tissue (including full thickness skin tissue). These or other MSCs can be used in aspects of the present disclosure. The MSCs can be generally an adherent cell population expressing markers CD90 and CD 105 (>90%) and lacking expression of CD34 and CD45 and MHC class II (<5%) as detected by flow cytometry.

The cells used in the embodiments herein can be from any suitable species of animal, for example a mammal, such as a human, canine (e.g. dog), feline (e.g. cat), equine (e.g. horse), porcine, ovine, caprine, or bovine mammal.

The cell composition used to prepare the trehalose-containing cell suspension can be combined with trehalose in a variety of ways. In certain modes, the cell composition can be combined with an aqueous liquid medium, such as an aqueous solution, containing the trehalose. In this regard, the cell composition can be added to the medium containing trehalose, the medium containing trehalose can be added to the cell composition, or both. The combination of the two materials can be conducted so as to preserve the viability of the cells to the extent practicable. A gradual combination, optionally with gentle agitation, can be conducted for these purposes.

When a cryopreserved cell composition is used, it is typically thawed prior to combining it with the trehalose-containing medium. Any suitable thawing technique can be used. In certain forms, the cell composition is thawed by immersion of a container, such as a bag or vial, containing the cryopreserved cells into a liquid bath, e.g. heated to about 37° C. After the cell composition has thawed (e.g. by the observation of a lack of ice crystals in the composition), the cell composition can be combined with the trehalose-containing medium. In some forms, the container for the cryopreserved cells is provided with a sterile access port or member, such as a septum, and after thawing, the thawed cell composition including the cells and the medium in which they were stored is drawn from the container with a needle and syringe or other suitable transfer device. At least the cell component thereof is thereafter combined with the trehalose-containing medium, and in some forms the cells and the storage medium are combined with the trehalose-containing medium. In cases where the storage medium is not included in the combination step with the trehalose-containing medium, the cells can for example be washed with another physiologically-acceptable medium, and then combined with the trehalo se-containing medium. In either case, the cell composition combined with the trehalose-containing medium can, and will typically, include cell components that have been released by dead cells into the liquid medium suspending the cells, including for example DNA. This DNA can be a contributing factor to the tendency of the cells to form clumps, and the presence of the trehalose in the prepared cell suspension can inhibit the DNA-facilitated clumping of the cells.

The combination of the cells (and optionally their storage or wash medium) with the trehalo se-containing medium can be conducted in any suitable container or vessel. In beneficial modes, the combination is conducted in a second container (other than the cryostorage or other container in which the cells were stored or held). This second container can include an input port or other input member, for example a septum, for sterile transfer of materials such as the cells and/or the trehalo se-containing medium into the second container. In some forms, combining the cell composition and trehalose-containing medium can include delivering both of these into the second container, for example in either order or simultaneously. In other forms, the second container can be provided as a pre-manufactured container already containing the trehalo se-containing medium in sterile condition, and the cell composition can be added to the pre-manufactured container. In any of these embodiments, the second container can be a bag and/or can have an outlet port spaced from the input port or other member for delivery of the cells from the bag or other container, e.g. for delivery into a patient. The second container can be a bag having a septum for sterile input of materials and a valved port for outlet of materials, e.g. as occurs in common saline bags for patient treatment in medical care. The cells (potentially with their storage or wash medium), and potentially the trehalo se-containing medium (if not pre-manufactured in the bag) can be sterilely delivered into the bag by needle through the septum, and the prepared trehalose-containing cell suspension can be sterilely delivered to the patient through the valved port.

The prepared trehalose-containing cell suspension can have any suitable density of the cells. In some embodiments the prepared trehalose-containing cell suspension will have a cell density of at least 100,000 cells/mL, at least 200,000 cells/mL, at least 500,000 cells/mL, at least 1 million cells/mL, or at least 2 million cells/mL. Typically the cell density in such prepared cell suspension will have a cell density in the range of 100,000 cells/mL to 100 million cells/mL, more typically in the range of 100,000 cells/mL to 10 million cells/mL, and even more typically 100,000 cells/mL to 5 million cells/mL. In certain forms the prepared trehalose-containing cell suspension can have a cell density in the range of about 0.5 million cells/mL to about 3 million cells/mL.

The prepared trehalose-containing cell suspension can have a suitable concentration of trehalose, desirably a concentration that inhibits clumping of cells in the cell suspension. In some embodiments the prepared trehalo se-containing cell suspension will have a trehalose concentration of at least about 0.5%, at least 1%, at least 2%, or at least 3% by weight. Typically the trehalose concentration in such prepared cell suspensions will be in the range of about 0.5% to about 20%, more typically in the range of about 1% to about 15%, more typically in the range of about 2% to about 10%, and in certain embodiments about 2.5% to about 8%. In certain forms the prepared trehalose-containing cell suspension will have a trehalose concentration of about 3% to about 4%. It has been discovered that even at relatively low or moderate concentrations as identified herein, trehalose can inhibit cell clumping in the prepared cell suspensions.

The prepared trehalose-containing cell suspensions can be put to any suitable use, including for example research or therapeutic uses. For therapeutic use, the cell suspension may as examples be administered to a human or animal patient to treat or prevent a disease or condition such as degenerative bone disease, osteoarthritis, rheumatoid arthritis, polyarthritis, systemic lupus erythematosus, inflammatory bowel disease, atopy, hepatitis, chronic steroid responsive meningitis-arteritis, beagle pain syndrome, degenerative myelopathy, chronic renal failure disease, dilated and mitral cardiomyopathy, keratoconjunctivitis sicca, immune mediated non-erosive arthritis, immune mediated hemolytic anemia, immune mediated thrombocytopenia, Evans syndrome, intervertebral disc disease, muscle fibrosis secondary to disease or trauma, refractory corneal ulcer, diabetes mellitus, spinal trauma, eosinophilic granuloma complex, hypertrophic cardiomyopathy, cholangitis, spinal injury, exercise induced pulmonary hemorrhage, rhabdomyolysis, corneal ulcer, eczema, multiple sclerosis, muscular dystrophy, spinal injury, diabetes mellitus, hepatitis, myocardial infarction, congestive heart failure, or muscle fibrosis.

The cell suspension can be administered to a patient in any suitable manner. In certain forms, the cell suspension is delivered systemically into the bloodstream of a patient, for example by delivery into a vein or artery. In other forms, the cell suspension is delivered topically to the patient (e.g. in the treatment of atopy or other skin disorders). In still other forms, the cell suspension is delivered to a local implant site in a patient. Any of these or any combination of these modes of administration may be used in the treatment of a patient. In certain combination treatments, a first amount of a trehalose-containing cell suspension herein can be delivered systemically into the bloodstream of a patient, and a second amount of a trehalose-containing cell suspension herein (e.g. prepared with or separately from the first amount and including the same type(s) or a different type(s) of cells) is implanted locally in or near one or more skeletal joints in a patient to treat an arthritic condition, e.g. any of those arthritic conditions identified herein. Also, in patient treatments herein, a single administration of a trehalose-containing cell suspension as described herein can be made in some embodiments, while in others multiple separate administrations of trehalose-containing cell suspensions as described herein may be made over time (e.g. weekly or monthly administrations). In further embodiments, the cell suspension can be filtered prior to administration to the patient, e.g. to remove any clumps of cells that may be present. In certain forms, the cell suspension can be passed through an in-line filter positioned in tubing through which the cell suspension is passed into the blood stream of the patient, e.g. into a vein or artery of the patient. Such a filter can, in certain variants, have a particle size cutoff of about 200 micrometers (i.e. exclude from passage particles having a maximum cross-sectional dimension of greater than about 200 micrometers) or lower, or a particle size cutoff of about 170 micrometers or lower, or a particle size cutoff of about 100 micrometers or lower, while allowing the passage of singly suspended cells through the filter.

Additional embodiments herein include products useful in preparing trehalose-containing cell suspensions as described herein. In one embodiment, provided is an aqueous medium containing trehalose useful for preparing trehalose-containing cell suspensions. The aqueous medium containing trehalose can contain those components, and in amounts, as specified herein. As well, the aqueous medium containing trehalose can be provided in sterile form in a container that is included in the kit. That container may be a vial, bag or other container. In certain forms, the container has the features of the “second container” discussed hereinabove in which the trehalose-containing cell suspension can be prepared, including for example having an inlet port or other member (e.g. needle septum) and a separate outlet port as discussed above. Kits disclosed herein may include the container containing the trehalose-containing aqueous medium along with one or more additional components, for example including but not limited to a liquid transfer device such as a syringe and attached or attachable needle, and potentially also a container containing the cell composition to be used to prepare the trehalose-containing cell suspension. The container containing the cell composition can include the composition in a cryopreserved state (e.g. shipped frozen with the kit) or in a non-cryopreserved (e.g. thawed where the cells were previously cryopreserved) state. Kits disclosed herein may also include at least one filter, for example a filter as described above, through which a prepared trehalose-containing cell suspension can be passed prior to administration into a patient, and/or tubing through which the cell suspension can be passed during administration to the patient.

The following specific Experimental is provided to facilitate a further understanding of aspects of the present disclosure. It will be understood that this Experimental is illustrative, and not limiting, in nature.

EXAMPLE 1

### 1.1 Summary

A study was conducted to compare the efficacy of dextrose, hydroxyethyl starch (VetStarch™) and trehalose to prevent the clumping of previously-cryopreserved canine uterine regenerative cells after thawing as compared to saline alone. Dextrose showed no reduction in cell clumping. Hydroxyethyl starch showed some improvement but nonetheless provided a preparation with medium clumps. It was discovered that trehalose greatly reduces clumping, with any clumps present being tiny generally spheroid particles barely visible to the human eye as compared to long, stringy structures in the saline sample. After exposure to trehalose for an estimated duration of cell infusion, cell viability remained above 90%. Further, cell morphology was normal when the cells were plated into T25 flasks.

### 1.2 Cells

The cells used in this Example were purified mesenchymal stem cell populations obtained from canine uterine tissue (from passage 5). The cells were frozen in cryovials in 2% DMSO in VetStarch. The frozen cell samples were provided as 2 mL aliquots with a cell density of 10 million cells/mL. Cells from 3 different canine donors were included n the testing, labeled herein as Donor1; Donor2 and Donor3.

### 1.3 Bag Preparation

Experiments were performed in 50 mL bags commonly used for saline solutions to be administered to the bloodstream of patients. The experimental volume for the prepared cell suspension was between 5-10 mL depending on the run. Chemicals tested for their anti-clumping ability included dextrose, hydroxyethyl starch, and trehalose. Concentrations of these chemicals varied between experiments. Solutions of anti-clumping agents were prepared then injected into the bags. Concentration and volumes were computed so that targeted concentrations of anti-clumping agents were reached once the cells were injected in the bag. Volumes were chosen so that the cells were at one million cells/mL after preparation of the final composition in the bags.

### 1.3 Cell Thawing and Injection

Cryovials containing the cells were thawed in a 37° C. water bath until ice crystals just thawed. Cells were drawn out of the vial through a needle septum of the vial with an 18 gauge needle and slowly added to the bag through a needle septum of the bag so as not to lyse the cells. The vial was then washed with saline and this wash volume was also carefully injected into the bag through the needle septum. The bags were then monitored and the condition of the cells was observed for between 10 to 80 minutes.

### 1.4 Post-Bag Evaluation of Cells

After time in the bag had expired, the cells were carefully removed from the bag with a syringe and 18 gauge needles. This volume was carefully placed in a 50 mL conical flask. It was mixed to ensure even distribution of cells, then mixed 1:1 with trypan blue and counted to determine viability. The volume was then centrifuged at 400 g for 5 minutes, supernatant aspirated, and resuspended in 2mL complete media. 1 mL of this cell suspension was then plated into a T25 flask. 24 hours later, the plates were observed and the condition of the cells noted.

### 1.5 Results—Comparison of Trehalose to Hydroxyethyl Starch

Clumping. Cells from Donor1 were exposed to candidate anti-clumping agents trehalose (3.3% in final preparation) and hydroxyethyl starch (25% in final preparation) in the bag at a cell concentration of 1 million/mL. For the trehalose sample, after 80 minutes, there were still no clumps formed. The solution was seen to be somewhat turbid but not clumpy. For the hydroxyethyl starch sample, clumping was observed in 12 minutes, with small but visible spheroid clumps formed.

Viability. At the end of the clumping experiment, cells were removed from the bag, tested for viability by trypan blue staining, and plated into T25 flasks. Cells in the trehalose exhibited a viability of 94% whereas cells in the hydroxyethyl starch exhibited a viability of 80%As seen in Table 2, cells in VetStarch were about 10% less viable compared to cells in trehalose.

Cell morphology. 24 hours after plating, the T25 flasks were observed. The cell morphology was normal for both the hydroxyethyl starch and the trehalose samples.

### 1.6 Results—Comparison of Trehalose to Dextrose

Cells from Donor2 were exposed to dextrose (5%) and trehalose (1.65% and 3.3%) and dextrose (5%) in the bag at a cell concentration of 1 million/mL. For the dextrose sample, small clumps began to form within 10 minutes and after 80 minutes there were many stringy clumps. For the 1.65% trehalose sample, small clumps began to form after 20 minutes and after 80 minutes there were a few stringy clumps. For the 3.3% trehalose sample, small clumps began to form after 20 minutes and after 80 minutes there were only several clumps and they remained small.

1.7 Results—Trehalose with Other Donor Cells

Cells from Donor1 and Donor3 were exposed to trehalose (3.3%) in the bag at a cell concentration of 1 million/mL. The Donor3 sample began to form some clumps after 20 minutes but they were very small and barely visible and after 80 minutes there were only several very small clumps and they were still barely visible. For the Donor1 sample, some clumping was observed at 20 minutes but again they were barely visible, very small clumps, and after 80 minutes essentially all clumps were very small and barely visible with the exception of one or two slightly larger clumps.

1. A method for preparing a cellular composition, comprising:
(a) thawing cryopreserved cells to provide thawed, viable cells; and
(b) combining the thawed, viable cells with an aqueous medium containing trehalose to provide a trehalose-containing cell suspension.

2. A method, comprising:
(a) suspending cells in a cryoprotectant-containing medium to form a cryopreservable cell composition;
(b) subjecting the cryopreservable cell composition to cryopreservation conditions to form cryopreserved cells;
(c) thawing the cryopreserved cells to provide a thawed, viable cells; and
(d) combining the thawed, viable cells with an aqueous medium containing trehalose to provide a trehalose-containing cell suspension.

3. A method for preparing a diluted cell suspension from a cell concentrate, comprising:
(a) thawing a cryopreserved cell composition having a first viable cell density of at least 3 million cells/mL to provide thawed, viable cells; and
(b) combining the thawed, viable cells with an aqueous medium containing trehalose to form a trehalose-containing cell suspension having a second viable cell density less than the first cell density.

4. The method of any preceding claim, wherein the aqueous medium has a trehalose concentration in the range of 1% to 10% by weight.

5. The method of any preceding claim, wherein the aqueous medium also contains about 0.9% by weight sodium chloride.

6. The method of any preceding claim, wherein the aqueous medium has an osmolarity in the range of 200 to 600 mosmol/kg.

7. The method of any preceding claim, wherein the cells are mammalian cells.

8. The method of any preceding claim, wherein the cells are human cells.

9. The method of any preceding claim, wherein the cells are stem cells.

10. The method of any preceding claim, wherein the cells are mesenchymal stem cells.

11. The method of claim 10, wherein the mesenchymal stem cells are derived from dental tissue, testicle tissue, bone marrow; peripheral blood, placental tissue, uterine tissue, umbilical cord blood, umbilical cord tissue, or skin tissue.

12. The method of any preceding claim, wherein the trehalose is present in an amount effective to reduce clumping of the cells in the trehalo se-containing cell suspension.

13. The method of any preceding claim, wherein said combining is conducted so as to provide a trehalose concentration in the range of about 1.5% to about 7% by weight in the trehalose-containing cell suspension.

14. The method of any preceding claim, wherein said combining includes the steps of:
removing the thawed cells from a cryopreservation container; and
combining the thawed cells with the aqueous medium containing trehalose in a second container.

15. The method of any preceding claim, also comprising administering the trehalose-containing cell suspension to a human or animal patient.

16. The method of any preceding claim, wherein the cells are canine, feline, equine, ovine, bovine, porcine, or human cells.

17. The method of any preceding claim, wherein said combining comprises combining a liquid medium containing the thawed, viable cells and also containing DNA released from dead cells with the aqueous medium containing trehalose.

18. The method of claim 17 wherein the liquid medium is a medium in which the cells had been cryopreserved.

19. A cellular composition prepared by or preparable by a method according to any preceding claim.

20. A cellular composition, comprising:
cells suspended in a liquid aqueous medium including trehalose; wherein the concentration of trehalose is at least 0.5% by weight of the composition.

21. The cellular composition of claim 20, wherein the trehalose is effective to inhibit clumping of the cells.

22. The cellular composition of claim 20 or 21, wherein the cells comprise mesenchymal stem cells.

23. A method according to any one of claims 1 to 18, also comprising filtering the trehalose-containing cell suspension.

24. A kit, comprising:
a liquid trehalose composition; and
a container for example combining the liquid trehalose composition with cells.

25. The kit of claim 24, also comprising a container containing cells to be combined with the liquid trehalose composition.

26. The kit of claim 24 or 25, also comprising a filter.

27. The method of any of claims 1 to 3, wherein the aqueous medium also contains about 0.9% by weight sodium chloride.

28. The method of any of claims 1 to 3, wherein the aqueous medium has an osmolarity in the range of 200 to 600 mosmol/kg.

29. The method of any of claims 1 to 3, wherein the cells are mammalian cells.

30. The method of any of claims 1 to 3, wherein the cells are human cells.

31. The method of any of claims 1 to 3, wherein the cells are stem cells.

32. The method of any of claims 1 to 3, wherein the cells are mesenchymal stem cells.

33. The method of claim 32, wherein the mesenchymal stem cells are derived from dental tissue, testicle tissue, bone marrow; peripheral blood, placental tissue, uterine tissue, umbilical cord blood, umbilical cord tissue, or skin tissue.

34. The method of any of claims 1 to 3, wherein the trehalose is present in an amount effective to reduce clumping of the cells in the trehalose-containing cell suspension.

35. The method of any of claims 1 to 3, wherein said combining is conducted so as to provide a trehalose concentration in the range of about 1.5% to about 7% by weight in the trehalose-containing cell suspension.

36. The method of any of claims 1 to 3, wherein said combining includes the steps of:
removing the thawed cells from a cryopreservation container; and
combining the thawed cells with the aqueous medium containing trehalose in a second container.

37. The method of any of claims 1 to 3, wherein said combining comprises combining a liquid medium containing the thawed, viable cells and also containing DNA released from dead cells with the aqueous medium containing trehalose.

38. The method of claim 37 wherein the liquid medium is a medium in which the cells had been cryopreserved.
